(19) United States
(12) Patent Application Publication
(10) Pub. No.: US 2004/0137546 A1
(43) Pub. Date: Jul. 15, 2004

(54) Method for determination of free and combined glycerin in biodiesel

(75) Inventor: Stacey J. Greenhill, Wheeling, IL (US)

Correspondence Address: MCANDREWS HELD & MALLOY, LTD, 500 WEST MADISON STREET, SUITE 3400, CHICAGO, IL, 60661

(73) Assignee: Stepan Company, Northfield, IL (US)

(21) Appl. No.: 10/744,272

(22) Filed: Dec. 22, 2003

Related U.S. Application Data
(60) Provisional application No. 60/436,332, filed on Dec. 24, 2002.

(51) Int. Cl. C12Q 1/66

(52) U.S. Cl. 435/8; 436/60

(57) ABSTRACT

A method for determining the glycerin content of a biodiesel sample is provided which utilizes the conversion of the glycerin content of the biodiesel sample into a colored compound, preferably quinoneimine dye. The concentration of the colored compound, preferably quinoneimine dye, in the biodiesel sample may then be measured and converted to a concentration of glycerin in the biodiesel sample.

### PRIORITY CLAIM

This application claims the benefit of U.S. Provisional Application No. 60/436,332, filed Dec. 24, 2002.

### BACKGROUND OF THE INVENTION

Biodiesel is defined as the mono alkyl esters of long chain fatty acids derived from vegetable oils or animal fats, for use in compression-ignition (diesel) engines. As such, government regulatory bodies have adopted strict specifications for biodiesel and in particular its free and bonded glycerin contents. The free and bonded glycerin contents of a biodiesel sample are the total glycerin content for a given biodiesel sample. A high free and/or total glycerin content in a biodiesel sample can result in substantial problems for the machinery employing the fuel. For example, high free and/or total glycerin content can result in substantial fouling of the injection nozzles and can also contribute to the formation of deposits on the injection nozzles, pistons, and valves of an engine. Accordingly, regulations typically specify a maximum free glycerin content of 200 ppm (0.020%) and a maximum total glycerin content of 2400 ppm (0.240%).

The biodiesel industry presently uses a standard method for glycerin determination—ASTM D 6584-00, “Test Method for Determination of Free and Total Glycerin in B-100 Biodiesel Methyl Esters By Gas Chromatography.” This standard method is a lengthy procedure involving a silylating agent and pyridine and is only applicable to methyl ester systems. The procedure typically takes about 5 to 7 hours. The use of pyridine raises substantial safety issues. Additionally, this standard method requires the use of expensive and delicate gas chromatography equipment. The equipment, in particular the gas chromatography column, has a limited life expectancy and requires constant calibration. Further, the results achieved using this standard method are difficult to reproduce from laboratory to laboratory.

A second approach for determining the glycerin content of a biodiesel sample has been proposed. This approach can be found at http://koal2.cop.fi/leonardo/fbglycerol.htm. This approach uses commercially available test kits obtained from Boehringer-Mannheim. The kits describe the method of glycerin determination in its product insert as follows:

“Glycerol is phosphorylated by adenosine-5′-triphosphate (ATP) to L-glycerol-3-phosphate in the reaction catalyzed by glycerol kinase (GK) (1).

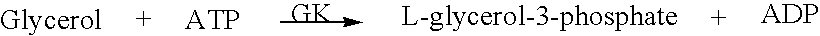

The adenosine-5′diphosphate (ADP) formed in the above reaction is reconverted into ATP by phosphoenolpyruvate (PEP) with the aid of pyruvate kinase (PK) with the formation of pyruvate (2).

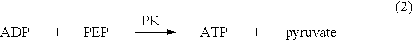

In the presence of the enzyme L-lactate dehydrogenase (L-LDH), pyruvate is reduced to L-lactate by reduced nicotinamide-adenine dinucleotide (NADH) with the oxidation of NADH to AND (3).

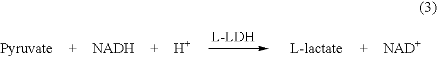

The amount of NADH oxidized in the above reaction is stoichiometric to the amount of glycerol. NADH is determined by means of its light absorption at 334, 340 or 365 nm.”

The Boehringer-Mannheim test kits are described for use in determining the glycerin in foodstuffs and cosmetics. The adaptation of the Boehringer-Mannheim test kits to a determination of free and total glycerin in a biodiesel sample as described in the above-identified website results in a cumbersome procedure that calls for various repetitive steps as well as separate solid phase extractions or liquid-liquid extractions some of which are very time-consuming.

Accordingly, there is a need for a faster, more reliable, and less costly method for determining both the free and total glycerin contents of a biodiesel sample.

### BRIEF SUMMARY OF THE INVENTION

The present invention is a method of determining both the free and total (combined free and bonded) glycerin contents of a biodiesel sample. The method of the present invention utilizes an enzymatic conversion of the glycerin present in the biodiesel sample into a colored compound, often a quinoneimine dye. Such enzymatic glycerin conversion systems have been known and used in the medical industry for the quantitative in vitro measurement of triglycerides in serum or plasma. The determination of serum or plasma triglycerides is useful in the diagnosis and treatment of patients with diabetes mellitus, liver obstruction, nephrosis and other diseases involving endocrine disorders and lipid metabolism. The general principles of this system were reported by P. Trinder. See, P. Trinder, “Determination of Glucose in Blood using Glucose Oxidase with an Alternative Oxygen Acceptor,” Ann. clin. Biochem., 6 (1969) 24; and D. Barham and P. Trinder, “An Improved Colour Reagent for the Determination of Blood glucose by the Oxidase System,” Analyst, February, 1972, Vol. 97, pp. 142-145. P. Trinder et al. report the relevant reactions as follows

Glucose+O₂+glucose oxidase+H₂O→gluconic acid+H₂O₂ (1)

H₂O₂+oxygen acceptor+peroxidase→coloured product. (2)

The amount of the colored compound or quinoneimine dye present in the sample after testing is typically directly proportional to the amount of the glycerin in the biodiesel sample. Additionally, the method of the present invention provides for converting mono-, di- and tri-glycerides into glycerin. After conversion of these constituents to glycerin, the enzymatic conversion of the glycerin to the colored compound or quinoneimine dye can be accomplished thereby providing in the test sample a concentration of colored compound or quinoneimine dye directly proportional to the total glycerin content of the biodiesel sample. The increase in absorbance at the appropriate wavelength (typically 490 to 560 nm, preferably 540 nm, for quinoneimine dye) is directly proportional to the glycerin concentration of the sample.

The preferred reaction by which the glycerin is converted to the colored compound may generally be described as follows:

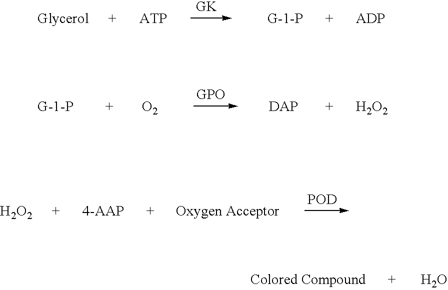

Where: GK=Glycerol Kinase, ATP=adenosine triphosphate, G-1-P=Glycerol-1-phosphate, ADP=Adenosine-05′-diphosphate, GPO=glycerol phosphate oxidase, DAP=dihydroxyacetone phosphate, 4-AAP=4-aminoantipyrine, and POD=peroxidase. The oxygen acceptor may be sodium N-ethyl-N-(3-sulfopropyl)m-anisidine (ESPA); 3,5-dichloro-2-hydroxybenzene sulfonate (DHBS); 3-hydroxy-2,4,6-tribromobenzoic acid (TBHB); 4-chlorophenol; 3,5-dichloro-2-hydroxybenzensulfonic acid (DCBS), N-ethyl-N-(2-hydroxy-3-sulfopropyl) toluidine (TOOS), or any other compatible compound that exhibits a change in color with oxidation.

### DETAILED DESCRIPTION OF THE INVENTION

The present invention provides a method to easily determine both the free and total glycerin content of a biodiesel sample. As noted above, the method of the present invention, also referred to as the Greenhill Method, utilizes an enzymatic conversion of glycerin into a colored compound, preferably quinoneimine dye. The concentration of the colored compound can then be determined by an absorbance measurement to determine the concentration of the glycerin in the biodiesel sample. The standard absorbance measurement for the preferred quinoneimine dye is about 540 nm. The preferred enzymatic reaction of the present invention may be described as follows.

Glycerol is phosphorylated by adenosine triphosphate (ATP) forming glycerol-1-phosphate (G-1-P) and adenosine-05′-diphosphate (ADP). This reaction is catalyzed by glycerol kinase (GK).

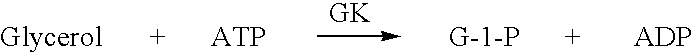

G-1-P is then oxidized by glycerol phosphate oxidase (GPO) to dihydroxyacetone phosphate (DAP) and hydrogen peroxide (H₂O₂).

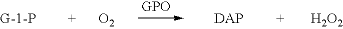

A colored compound, typically a quinoneimine dye, is produced by the peroxidase (POD) catalyzed coupling of 4-aminoantipyrine (4-AAP) and an oxygen acceptor with H₂O₂. The oxygen acceptor may be sodium N-ethyl-N-(3-sulfopropyl)m-anisidine (ESPA); 3,5-dichloro-2-hydroxybenzene sulfonate (DHBS); 3-hydroxy-2,4,6-tribromobenzoic acid (TBHB); 4-chlorophenol; 3,5-dichloro-2-hydroxybenzensulfonic acid (DCBS); N-ethyl-N-(2-hydroxy-3-sulfopropyl) toluidine (TOOS); or any other compatible compound that exhibits a change in color with oxidation. The concentration of the colored compound can be determined by standard absorbance techniques and will typically correspond directly to the concentration of the glycerol in the sample. For example, for those oxygen acceptors that generate a quinoneimine dye, the concentration of the quinoneimine dye may be determined at an absorbance maximum at about 500 nm to about 550 nm, preferably 540 nm. The increase in absorbance is typically directly proportional to the glycerin concentration of the sample.

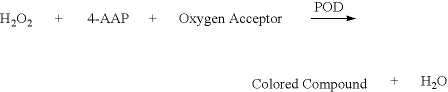

This reaction pathway will be useful for measuring the glycerin content of the biodiesel sample. As a proposed initial step prior to subjecting the biodiesel sample to the above-described reactions, in order to determine the total glycerin content of the biodiesel sample, one may convert any bonded glycerin, such as mono-, di-, or triglycerides, into glycerin. After the conversion of the bonded glycerin into free glycerin, the above reaction pathway will be useful for measuring not only the free glycerin content of the biodiesel sample but also the total glycerin content of that sample.

The proposed method of converting the bonded glycerin into free glycerin of a biodiesel sample is to subject the bonded glycerin to a saponification with potassium hydroxide. This reaction can be described as follows.

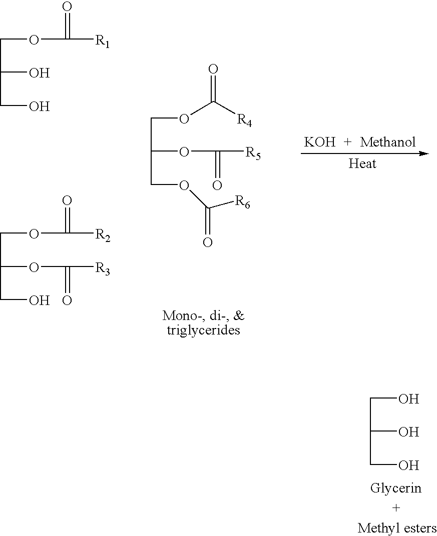

Where R₁, R₂, R₃, R₄, R₅, and R₆ may be the same or different and an aliphatic chain typically found in vegetable or animal oil sources, typically C₈ to C₂₂, the chains may be saturated or unsaturated.

The procedure for determining the total glycerin content of a biodiesel sample may proceed as follows. First, the bonded glycerin, if any in the sample, may be converted to glycerin by taking a known volume of the biodiesel sample, preferably 5 μl to 1000 μl, most preferably 100 μl, into a standard container, preferably a 5 ml centrifuge tube. To this biodiesel sample may be added a known volume of caustic, preferably KOH, most preferably 1 ml 1N in methanol, and the solution mixed well. The solution may then be heated, preferably in a hot water bath at about 70-80° C. so that the sample temperature reaches 70-80° C. for 15 to 60 minutes, most preferably about 30 minutes. An oven may also be used to heat the samples to the appropriate temperature of about 70-80° C. for 15 to 60 minutes. A known volume of acid, preferably HCl, most preferably 1 ml 1N, may then be added to neutralize the sample and the sample mixed well. Petroleum ether or hexane may then be added, preferably approximately 2 ml, to assist in phase separation, and the sample again mixed well. The phase separation may be assisted by placing the sample in a centrifuge, preferably for between about 1 to 15 minutes, preferably about 5 minutes. The bottom phase will include the total glycerin of the biodiesel sample. Any conventional device may be used to extract a known volume of the lower layer, preferably 25 μl to 1000 μl, most preferably 250 μl, of this lower phase of the sample for further processing in the enzymatic conversion aspect of the method of the present invention. This step of converting the bonded glycerin of the biodiesel sample to free glycerin may be referred to as the saponification step.

Turning now to the enzymatic conversion of the glycerin of the biodiesel sample, the biodiesel sample may be subjected to the enzymatic conversion of its glycerin content to the colored compound, preferably a quinoneimine dye, as follows. A known volume, preferably 25 μl to 1000 μl, most preferably 250 μl, sample from the lower phase from the saponification step (or 5 μl to 500 μl, preferably 50 μl, if one is simply measuring the free glycerin content and did not undertake the saponification step) may be placed into a container, preferably a 10 ml cuvette. To this lower phase may be added a known volume of a mixture containing the enzymatic reagents that perform the conversion of the glycerin to the colored compound. Preferably, the enzymatic reagent mixture includes an inorganic metal salt which is believed to promote phase separation and operate as a co-catalyst with the glycerol kinase.

The preferred enzymatic reagent mixture is commercially sold by Sigma Aldrich, St. Louis, Mo., under the trade name GPO Trinder A Reagent. The Sigma Aldrich GPO Trinder A Reagent utilizes sodium N-ethyl-N-(3-sulfopropyl)m-anisidine (ESPA) as its oxygen acceptor. Other contemplated enzymatic reagent products are available from Raichem, Division of Hemagen Diagnostics, Inc., San Diego, Calif. (using either ESPA or 3,5-dichloro-2-hydroxybenzene sulfonate (DHBS) as the oxygen acceptor); JAS Diagnostics, Inc., Miami, Fla. (using DHBS or 3-hydroxy-2,4,6-tribromobenzoic acid (TBHB) as the oxygen acceptor); Pointe Scientific, Inc., Lincoln Park, Mich. (using 4-chlorophenol as the oxygen acceptor); Valtek Diagnostics, Santiago, Chile (using 3,5-dichloro-2-hydroxybenzensulfonic acid (DCBS) as the oxygen acceptor); Catachem, Inc. Bridgeport, Conn. (using N-ethyl-N-(2-hydroxy-3-sulfopropyl) toluidine (TOOS) as the oxygen acceptor). Of course, those skilled in the art will appreciate that these commercial enzymatic reagent mixture are merely examples and any similar formulations, commercial or non-commercial, may be equally effective in the method of the present invention. At present, those enzymatic reagent mixtures that do not include lipase (such as the mixture available from Sigma Aldrich) are preferred.

The components and concentrations of Sigma Aldrich's GPO Trinder A Reagent mixture when reconstituted for use in the preferred method of the present invention are as follows.

ATP, 0.250 mmol/L

Magnesium salt, 2.50 mmol/L

Sodium-N-ethyl-N(3-sulfopropyl) m-anisidine (ESPA), 0.125 mmol/L

Glycerol kinase, 0.083 U/L

Glycerol phosphate oxidase 1667 U/L

Peroxidase, 1667 U/L

Buffer, pH 7.0

Nonreactive stabilizers and fillers, sodium azide, 0.03% added as preservative.

After adding the preferred enzymatic reagent mixture to the sample, the sample may be gently mixed, preferably with a vortex mixer. The sample may then be incubated in a water bath, preferably at about 32-37° C., or in an oven, preferably at about 50° C., such that the sample is heated to a temperature of about 32-37° C. for about 10 to 30 minutes, preferably about 10 to 15 minutes. After this heating step, if necessary, the sample may then be mixed again. Finally, the absorbance of the sample may be measured, by spectrometer at 490 nm to 560 nm, preferably at 540 nm, to determine the concentration of the quinoneimine dye of the sample. To insure that the measurement is not affected by time degradation, the spectrometer reading should be conducted within about 45 minutes, preferably within about 30 minutes, of the time the sample is removed from the bath or oven.

If no bath or oven is available, the reaction may be allowed to proceed at room temperature but may require more time. In such cases, the sample may be permitted to sit for about 15 to 60 minutes, preferably about 30 minutes, at 25° C.

Because the enzymatic conversion in the preferred embodiment results in a conversion of the glycerin of the biodiesel sample into quinoneimine dye, a simple method of determining the glycerin concentration of the sample is to determine the quinoneimine dye concentration of the sample. This may be accomplished easily by an absorbance reading in a standard spectrometer capable of reading absorbance at 540 nm. Accordingly, a standard absorbance reading and a blank absorbance reading may be undertaken on the spectrometer at 540 nm for comparison with the absorbance reading of the sample. In this regard, a glycerin standard, preferably 0.005% to 0.100% (50-1000 ppm), most preferably 400 ppm, may be prepared in a known volume, preferably 50 μl. To this volume may be added a known quantity of water, preferably 50 μl or 250 μl (depending on sample size). A blank standard of 50 or 250 μl water (depending on sample size) may also be prepared. As further described below, the blank standard is preferably comprised of deionized water and a known volume of the enzymatic reagent mixture.

The concentration of the quinoneimine dye of the sample may then be compared to the measurements made for the standard and the blank. The relationship between the concentration of quinoneimine dye and the concentration of glycerin content of the biodiesel sample is linear and may be extrapolated using the measurements for the known sample and the blank.

The above method has been described both generally and with reference to the preferred volumes, temperatures, times, and other factors. It will understood by those skilled in the art that any combination of such factors may readily be adapted to provide the method of the present invention.

The Sigma Aldrich Trinder A Reagent is preferred because the Trinder A Reagent is separate from other reagents, such as lipase, that are commonly also found in similar commercially-available enzymatic glycerin conversion products. Thus, other reagents, commercial or non-commercial, that include only the Trinder A Reagent would also be preferred.

Although GPO Trinder A is the preferred reagent, it is not always available. Alternative commercially-available enzymatic glycerin conversion products are available. A combined Trinder reagent, GPO Trinder, is a very common form of the combined reagent containing lipase. Because the lipase reagent is commonly used to convert the bound glycerin in biological samples to free glycerin for a determination of total glycerin in a biological sample, the lipase reagent may give a high result for free glycerin in a biodiesel sample because the lipase may saponify some of the mono-, di-, and triglycerides that constitute the bound glycerin (yielding an erroneously higher free glycerin measurement). If GPO Trinder A is not available, an alternative procedure for free glycerin determination is as follows using the combined reagent. The following procedure is also suitable for difficult samples, such as, but not limited to, in-process samples, “dirty samples,” or samples containing soap stock and/or high levels of fatty acids.

A 0.1-3.0 gram sample, most preferably 0.5 gram sample is weighed into a suitable container, most preferably a 10 mL centrifuge tube. A known amount of 50/50 alcohol and water, where the alcohol is methanol, ethanol, and/or isopropanol, most preferably 5 mL, is added to the container. A non-polar extraction solvent (1-5 ml), such as but not limited to petroleum ether, hexane(s), heptane, is added to the sample and the sample mixed well. It is beneficial to use a centrifuge to separate the polar (bottom alcohol/water) and non-polar (top organic) layers, although they will separate upon standing. The free glycerin will be in the polar (bottom alcohol/water) layer.

An aliquot of the bottom polar (bottom alcohol/water) layer is sampled, most preferably 0.05 mL (50 μL) and analyzed for glycerin as mentioned above.

Sample weight is calculated as follows:

Weight of biodiesel in aliquot=(biodiesel sample weight/lower layer volume)×aliquot volume.

The calculation is as follows for a 0.5 gram biodiesel sample, dissolved in 5.0 mL of alcohol/water and a 0.05 mL sample aliquot taken.

Weight of biodiesel in aliquot=(0.50/5.0)×0.05=0.0050 g.

The calculation for free glycerin is now as follows:

$$\begin{matrix}\begin{matrix}F . G . \\ ( ppm )\end{matrix} = \frac{( Abs Sample - Abs Blank )}{( Abs Standard - Abs Blank )} \times \\ \frac{( Conc . Standard \times Aliquot volume )}{Weight of biodiesel in aliquot}\end{matrix}$$

Where F.G. means free glycerin and Abs means absorbance.

An alternative embodiment of the present invention employs a slightly altered enzymatic conversion of the glycerin present in the biodiesel sample from that already described above. The alternative embodiment may generally be described as follows:

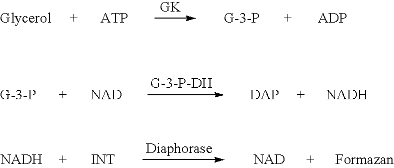

The glycerol is phosphorylated by adenosine triphosphate (ATP) to glycerol-3-phosphate (G-3-P) in a reaction catalyzed by the enzyme glycerol kinase (GK). In the presence of nicotinamide adenine dinucleotide (AND), G-3-P is oxidized by the enzyme glycerol-3-phosphate dehydrogenase (G-3-P-DH) to give dihydroxyacetone phosphate (DAP) and reduced AND (NADH). The NADH produced in this reaction reduces an equivalent amount of iodonitrotetrazolium violet (INT) in a reaction catalyzed by the enzyme diaphorase. The resulting formazan is an intensely colored dye. The absorbance of this compound at 500 nm is directly proportional to the original concentration of glycerol in the sample. An enzymatic reagent mixture that utilizes this mechanism is commercially available from Raichem and other commercial sources.

The particularly preferred method is set forth in the following procedure and has been found to be highly reproducible and accurate.

### Procedures

### Free Glycerin Determination (Procedure A)

Free glycerin content is determined using this procedure. Biodiesel samples, a blank (deionized water), and standard glycerin solutions may be transferred to cuvettes, preferably test tube style cuvettes. The prepared GPO-Trinder Reagent is added to each of the cuvettes (alternatively, the samples, standard solution, and blank may be added to cuvettes that already contain the GPO-Trinder reagent). The four solutions are mixed and gently heated. After heating, the absorbance of the samples, standard, and blank are measured at 540 nm and the glycerin content is calculated. (Note: For biodiesel samples requiring both free and total glycerin analyses, it is suggested, in order to save time, to start the saponification process (Procedure B, steps 1-3) prior to the free glycerin analysis.)

1. Turn the spectrometer on for a minimum of 15 minutes prior to analysis and set the wavelength to 540 nm. Zero the spectrometer using a cuvette filled with deionized water.

2. Remove the prepared GPO-Trinder Reagent from the refrigerator and allow it to warm to room temperature. If necessary, prepare fresh reagent. Quantitatively transfer the reagent into a suitable glass container, preferably a brown glass container, using an appropriate amount of deionized water and mix thoroughly. The prepared GPO-Trinder Reagent should be at room temperature prior to analysis and should have the following components in the following concentrations.

ATP, 0.250 mmol/L

Magnesium salt, 2.50 mmol/L

Sodium-N-ethyl-N(3-sulfopropyl) m-anisidine, 0.125 mmol/L

Glycerol kinase, 0.083 U/L

Glycerol phosphate oxidase 1667 U/L

Peroxidase, 1667 U/L

Buffer, pH 7.0

Nonreactive stabilizers and fillers, sodium azide, 0.03% added as preservative.

3. Label the cuvettes. For each sample set, cuvettes for two samples, one blank, and one standard sample are required. A deionized water blank and standard must be prepared for each set of samples.

4. Using a dispenser, preferably a pipette, most preferably a positive displacement micropipette, pipette 50 μl (0.050 ml) of the sample, glycerin standard, or deionized water blank into separate, labeled, cuvettes. (Note: To prevent contamination, a new micropipette tip should be used to transfer samples, glycerin standard, deionized water blank, GPO-Trinder Reagent, or water into the cuvettes. This is especially important when transferring the glycerin standard and deionized water blank. Alternatively, separate pipettes can be used for samples, glycerin standard, and the blanks.)

5. Transfer 3 ml of the GPO-Trinder Reagent to each cuvette, a 3 ml pipettor has been useful.

6. In the cuvettes, gently mix the samples, standard, and deionized water blank on the vortex mixer. Do not shake the cuvettes vigorously for an emulsion will be formed, which will lead to high glycerin results. If an emulsion is formed, discard the sample.

7. Place the cuvettes in a 50° C. oven or 32-37° C. water bath to heat the samples to 32-37° C., and set the timer for 10-15 minutes. Remove the cuvettes and mix again using the vortex mixer.

8. Read and record the absorbance at 540 nm for the deionized water blank and glycerin standard followed by each of the samples. The spectrometer does not need to be set to zero for the blank. The blank value is factored into the calculation. (Alternatively, the spectrometer may be set to zero for the blank; those skilled in the art will understand how to modify the associated calculations accordingly.)

9. Calculate the free glycerin content using Equation 1, Calculations section.

### Total Glycerin Determination (Procedure B)

This procedure may be used to determine total glycerin content (free and bonded) in biodiesel. Bonded glycerin is the glycerin portion of conjugated glycerides. The biodiesel sample is saponified using methanolic potassium hydroxide. After heating, the caustic solution is neutralized with aqueous hydrochloric acid and extracted with a non-polar extraction solvent, preferably petroleum ether, hexane or heptane, most preferably petroleum ether, to remove the methyl esters. The sample is centrifuged to separate the aqueous/alcohol and organic layers and a portion of the aqueous/alcohol (lower) layer is sampled for free glycerin (Procedure A) analysis. This procedure should be run in duplicate.

1. Use a micropipette to transfer 100 μl (0.1 ml) of the biodiesel sample into a labeled 5 ml centrifuge tube.

2. Add 1,000 μl (1 ml) of the standard 1.0 N methanolic KOH solution to the centrifuge tube using a micropipette. Cap tightly and mix thoroughly.

3. Place the closed centrifuge tubes in a water bath set at about 70-75° C. A metal rack may be used to support the samples. Heat the samples for 30 minutes.

4. Remove the centrifuge tubes. The samples should be fluid and a clear, yellow color. Discard any samples that appear to have “dried out”.

5. Using a fresh tip with the micropipette, pipette 1,000 μl (1 ml) of the standard 1.0 N HCl solution to each of the centrifuge tubes and mix thoroughly.

6. Add approximately 2 ml of petroleum ether to each of the centrifuge tubes and mix thoroughly. Place the tubes in the centrifuge and spin until separated, approximately 2-5 minutes.

7. Remove the tubes from the centrifuge. There should be two distinct layers.

Remove a 250 μl (0.25 ml) aliquot from the lower aqueous/alcohol layer, using a micropipette, and transfer to a labeled cuvette. (Note: Be sure that the lower layer is sampled and that no solid material is transferred to the cuvette. The best method for sampling the saponified samples is to press the plunger down so that the rubber tip is exposed before the solution is sampled.) (Note: Smaller sample aliquots of the saponified solution may be used; standard and blank solutions should be adjusted accordingly.)

8. Label blank and standard cuvettes.

Add 250 μl (0.25 ml) of deionized water to the blank cuvette. Add 50 μl (0.05 ml) of the glycerin standard and 200 μl (0.2 ml) of deionized water to the standard cuvette.

9. Pipette 3 ml of the GPO-Trinder Reagent into each cuvette with the pipettor and mix on the vortex mixer.

10. Continue the analysis from Procedure A, steps 7-8. Calculate the total glycerin content using Equation 2, Calculations section.

$$\begin{matrix}Calculations \\ ( Free Glycerin ) \\ F . G . ppm ( w \text{/} w ) = \frac{( A - B )}{( E - B )} \times \frac{C}{D} \\ \begin{matrix}\begin{matrix}A = Absorbance of Sample \\ B = Absorbance of Deionized Water Blank \\ C = Concentration of Glycerin Standard ( ppm ) \\ D = Density of Biodiesel ( 0.8348 gm \text{/} ml ) [ It may be necessary \\ to determine the biodiesel density on particular products \\ especially if the testing is done on ethyl ester , isopropyl \\ esters or blends . ] \\ E = Absorbance of Glycerin Standard\end{matrix} \\ \end{matrix} & Equation 1. \\ \begin{matrix}( Total Glycerin ) \\ T . G . ppm ( w \text{/} w ) = \frac{( A - B )}{( E - B )} \times \frac{F}{D} \\ \begin{matrix}A = Absorbance of Sample \\ B = Absorbance of Deionized Water Blank \\ D = Density of Biodiesel ( 0.8348 gm \text{/} ml ) \\ E = Absorbance of Glycerin Standard \\ F = Factor \\ F = \frac{C \times G}{H} \\ \begin{matrix}C = Concentration of Glycerin Standard ( ppm ) \\ G = Volume of Glycerin Standard ( 0.050 ml ) \\ H = Sample size ( ml ) \\ 0.0125 ml\end{matrix}\end{matrix} \\ H ( Sample size ) is calculated as follows : \\ H = \frac{0.1 ml \times Aliquot volume ( 0.25 ml )}{2 ml Total Volume}\end{matrix} & Equation 2. \\ ( Bonded Glycerin ) \\ \begin{matrix}Bonded Glycerin = Total Glycerin - \\ Free glycerin .\end{matrix} & Equation 3.\end{matrix}$$

These procedures set forth the preferred Greenhill Method. To demonstrate the accuracy and the reproducibility of the method of the present invention, samples were prepared and tested according to the preferred Greenhill Method and the ASTM D 6584-00 “Test Method for Determination of Free and Total Glycerin in B-100 Biodiesel Methyl Esters By Gas Chromatography.” The results demonstrate that the Greenhill Method is far superior in both accuracy and reproducibility to the ASTM D 6584-00 method.

Table I reflects the results obtained using the ASTM D 6584-00 method for both free and total glycerin evaluations of thirteen biodiesel samples. The listed samples were forwarded to two different laboratories that routinely test the glycerin content of biodiesel samples and are regularly contracted by biodiesel producers to measure the glycerin content of their biodiesel products to determine whether the biodiesel products meet government or other limits. All measurements are in ppm.

TABLE I
(Prior Art)

| | FREE GLYCERIN | | | TOTAL GLYCERIN* | | |
|---|---|---|---|---|---|---|
| | LAB 1 | LAB 2 | Δ | LAB 1 | LAB 2 | Δ |
| | RE- | RE- | between | RE- | RE- | between |
| SAMPLE | SULTS | SULTS | labs | SULTS | SULTS | labs |
| 1 | 60 | 190 | 130 | 100 | 210 | 110 |
| 2 | 40 | 50 | 10 | 270 | 80 | 190 |
| 3 | 150 | 360 | 210 | 280 | 590 | 310 |
| 4 | 170 | 340 | 170 | 390 | 560 | 170 |
| 5 | 370 | 420 | 50 | 470 | 620 | 150 |
| 6 | 200 | 160 | 40 | 210 | 3.70% | |
| 7 | 360 | 270 | 90 | 380 | 460 | 80 |
| 8 | 290 | 40 | 250 | 350 | 210 | 140 |
| 9 | 110 | 130 | 20 | 120 | 170 | 50 |
| 10 | 260 | 300 | 40 | 430 | 500 | 70 |
| 11 | 250 | 180 | 70 | 250 | 400 | 150 |
| 12 | 30 | 80 | 50 | 170 | 1620 | 1450 |
| 13 | 250 | 270 | 20 | 330 | 2120 | 1790 |

*The total glycerin is the sum of the free and bonded glycerin

As is evident from the results reported by the two laboratories as reflected in Table I, the ASTM D 6584-00 method has substantial problems in both accuracy and reproducibility. Particularly concerning is that the results for a given sample may be found to conform to government or other limits but if tested at a different laboratory the results indicate that the same sample does not conform to those same limits.

Table II demonstrates the consistent results that can be obtained by the Greenhill Method. In particular, four different analysts conducted the testing of 5 different samples for both free glycerin (Table IIa) and total glycerin (Table IIb). The consistent results returned by each of the analysts for each of the samples confirm that the Greenhill Method is both more accurate and more reproducible than the ASTM D 6584-00 method. All measurements are in ppm.

TABLE IIa
FREE GLYCERIN

| | Analyst 1 | | Analyst 2 | | Analyst 3 | | Analyst 4 | |
|---|---|---|---|---|---|---|---|---|
| | Trial | Trial | Trial | Trial | Trial | Trial | Trial | Trial |
| Sample | 1 | 2 | 1 | 2 | 1 | 2 | 1 | 2 |
| 1 | 132 | 135 | 136 | 133 | 130 | 136 | 137 | 131 |
| 2 | 18 | 20 | 31 | 22 | 24 | 23 | 6 | 3 |
| 3 | 133 | 131 | 140 | 137 | 137 | 135 | 131 | 130 |
| 4 | 12 | 13 | 22 | 20 | 25 | 24 | 11 | 9 |
| 5 | 36 | 33 | 49 | 48 | 46 | 38 | 34 | 36 |

TABLE IIb
TOTAL GLYCERIN*

| | | | |
|---|---|---|---|
| Analyst 1 | Analyst 2 | Analyst 3 | Analyst 4 |

| | Trial | Trial | Trial | Trial | Trial | Trial | Trial | Trial |
|---|---|---|---|---|---|---|---|---|
| Sample | 1 | 2 | 1 | 2 | 1 | 2 | 1 | 2 |
| 1 | 141 | 130 | 145 | 145 | 116 | 118 | 152 | 135 |
| 2 | 3480 | 3480 | 3395 | 3438 | 3661 | 3107 | 3240 | 3284 |
| 3 | 169 | 169 | 139 | 139 | 130 | 125 | 173 | 211 |
| 4 | 941 | 941 | 917 | 944 | 894 | 908 | 908 | 905 |
| 5 | 2050 | 2026 | 2102 | 2129 | 1931 | 1950 | 2037 | 1994 |

*The total glycerin is the sum of the free and bonded glycerin.

Table III tabulates the standard deviation and percent relative standard deviation for the results set forth in Tables IIa and IIb.

TABLE III

| | FREE GLYCERIN | | | TOTAL GLYCERIN* | | |
|---|---|---|---|---|---|---|
| | | | % Rel. | | | % Rel. |
| SAMPLE | Avg. | St. Dev. | Std. Dev. | Avg. | St. Dev. | Std. Dev. |
| 1 | 134 | 2 | 2 | 135 | 12 | 9 |
| 2 | 18 | 9 | 48 | 3386 | 160 | 5 |
| 3 | 134 | 3 | 2 | 157 | 27 | 17 |
| 4 | 17 | 6 | 35 | 920 | 18 | 2 |
| 5 | 40 | 6 | 15 | 2027 | 64 | 3 |

*The total glycerin is the sum of the free and bonded glycerin.

Table IV demonstrates the high accuracy of the Greenhill Method in detecting the glycerin content of a biodiesel sample. A stock biodiesel sample with a known glycerin concentration was spiked with various amounts of glycerin. The Greenhill Method was used to determine the glycerin content of each of these samples. Table IV sets forth the results of this testing. The samples were spiked with 98% GMS (glycerol mono stearate) and the “expected” column was calculated by taking the stock glycerin content of 155 and adding the amount of the glycerin spike added. All measurements are in ppm.

| | Glycerin | | Average | Recovery | Average | Recovery |
|---|---|---|---|---|---|---|
| | spike, | Ex- | Day | Day | Day | Day |
| Sample | ppm | pected | 1 | 1 | 2 | 2 |
| Stock | 0 | | 155 | | 158 | |
| 1 | 976 | 1131 | 1135 | 100.4% | 1104 | 97.6% |
| 2 | 1920 | 2075 | 2068 | 99.7% | 2030 | 97.9% |
| 3 | 2920 | 3075 | 3040 | 98.9% | 3002 | 97.6% |
| 4 | 3663 | 3818 | 3780 | 99.0% | 3774 | 98.8% |

As is evident from the results shown in Tables IIa, IIb, III, and IV, the Greenhill Method is highly accurate and reproducible.

The invention has been described with reference to preferred and alternate embodiments. Modifications and alterations will occur to others upon the reading and understanding of the specification. It is intended to include all such modifications and alterations insofar as they come within the scope of the appended claims or equivalents thereof.

1. A method of determining the glycerin content of a biodiesel sample comprising the steps of
providing a biodiesel sample;
subjecting the biodiesel sample to a reagent mixture whereby a colored compound is produced;
measuring the concentration of the colored compound in the biodiesel sample;
converting the measured concentration of colored compound of the biodiesel sample into a concentration of glycerin content of the biodiesel sample.

2. The method of claim 1 wherein the colored compound is a quinoneimine dye.

3. The method of claim 2 wherein the glycerin content of the biodiesel sample is converted into the quinoneimine dye with an enzymatic reagent mixture.

4. The method of claim 2 wherein the enzymatic reagent mixture comprises an oxygen acceptor.

5. The method of claim 2 wherein the oxygen acceptor is sodium N-ethyl-N-(3-sulfopropyl)m-anisidine (ESPA).

6. The method of claim 5 wherein the enzymatic reagent mixture further comprises glycerol kinase, glycerol phosphate oxidase, peroxidase, 4-aminoantipyrine (4-AAP), and adenosine triphosphate.

7. The method of claim 6 wherein the enzymatic reagent mixture further comprises an inorganic metal salt.

8. The method of claim 1 wherein the reagent mixture includes an oxygen acceptor and the colored compound is produced by oxidation of the oxygen acceptor.

9. The method of claim 8 wherein the oxygen acceptor is selected from the group consisting of sodium N-ethyl-N-(3-sulfopropyl)m-anisidine (ESPA); 3,5-dichloro-2-hydroxybenzene sulfonate (DHBS); 3-hydroxy-2,4,6-tribromobenzoic acid (TBHB); 4-chlorophenol; 3,5-dichloro-2-hydroxybenzensulfonic acid (DCBS); and N-ethyl-N-(2-hydroxy-3-sulfopropyl) toluidine (TOOS).

10. A method of determining the glycerin content of a biodiesel sample comprising the steps of
providing a biodiesel sample;
converting any combined glycerin in the biodiesel sample into glycerin;
converting the glycerin content of the biodiesel sample into a quinoneimine dye;
measuring the concentration of the quinoneimine dye in the biodiesel sample;
converting the measured concentration of quinoneimine dye of the sample into a concentration of glycerin content of the biodiesel sample.

11. The method of claim 10 wherein the combined glycerin is converted into glycerin via saponification.

12. The method of claim 11 wherein the saponification is completed using potassium hydroxide.

13. The method of claim 10 wherein the glycerin content of the biodiesel sample is converted into the quinoneimine dye with an enzymatic reagent mixture.

14. The method of claim 13 wherein the enzymatic reagent mixture comprises adenosine triphosphate.

15. The method of claim 14 wherein the enzymatic reagent mixture further comprises glycerol kinase, glycerol phosphate oxidase, and peroxidase, 4-aminoantipyrine (4-AAP) and sodium N-ethyl-N-(3-sulfopropyl)m-anisidine (ESPA).

16. The method of claim 15 wherein the enzymatic reagent mixture further comprises an inorganic metal salt.

17. A method of determining the glycerin content of a biodiesel sample comprising the steps of
providing a biodiesel sample;
converting the glycerin content of the biodiesel sample into a quinoneimine dye with an enzymatic reagent mixture, the enzymatic reagent mixture comprising adenosine triphosphate, glycerol kinase, glycerol phosphate oxidase, and peroxidase, 4-aminoantipyrine (4-AAP) and an oxygen acceptor;
measuring the concentration of the quinoneimine dye in the biodiesel sample;
converting the measured concentration of quinoneimine dye of the sample into a concentration of glycerin content of the biodiesel sample.

18. The method of claim 17 wherein the oxygen acceptor is selected from the group consisting of sodium N-ethyl-N-(3-sulfopropyl)m-anisidine (ESPA); 3,5-dichloro-2-hydroxybenzene sulfonate (DHBS); 3-hydroxy-2,4,6-tribromobenzoic acid (TBHB); 4-chlorophenol; 3,5-dichloro-2-hydroxybenzensulfonic acid (DCBS); and N-ethyl-N-(2-hydroxy-3-sulfopropyl) toluidine (TOOS).
